(19) United States
(12) Patent Application Publication
(10) Pub. No.: US 2024/0082050 A1
(43) Pub. Date: Mar. 14, 2024

(54) Systems, Methods and Apparatus for Application of Targeted Temperature Management Therapy Utilizing Concentric In/Out Cable

(71) Applicant: C. R. Bard, Inc., Franklin Lakes, NJ (US)

(72) Inventors: Gabriel A. Johnston, Broomfield, CO (US); Jesse Smith, Broomfield, CO (US); Mengjia Yi, Westminster, CO (US); Niharika Pathare, Thane (IN); Sean E. Walker, Platteville, CO (US)

(21) Appl. No.: 18/274,415

(22) PCT Filed: Jan. 26, 2022
(86) PCT No.: PCT/US2022/013904

§ 371 Date: Jul. 26, 2023

Related U.S. Application Data
(60) Provisional application No. 63/141,695, filed on Jan. 26, 2021.

(51) Int. Cl. A61F 7/00

(52) U.S. Cl. CPC A61F 7/0085; A61F 2007/0054; A61F 2007/0096

(57) ABSTRACT

Disclosed herein are systems and methods for providing targeted temperature management (TTM) therapy to a patient. For example, TTM systems include a connection system for coupling a fluid deliver line to a thermal contact pad. The connection system is configured to provide indication to the user that the fluid deliver line is completely connected to thermal contact pad. In addition, the connection system also includes a controller for activating a connection lock and for sharing signals with a TTM system module. The fluid deliver line includes a pair of conduits arranged concentrically and the thermal pad includes a TTM fluid filter disposed within a fluid containing layer.

### PRIORITY

This application claims the benefit of priority to U.S. Provisional Application No. 63/141,695, filed Jan. 26, 2021, which is incorporated by reference in its entirety into this application.

### BACKGROUND

The effect of temperature on the human body has been well documented and the use of targeted temperature management (TTM) systems for selectively cooling and/or heating bodily tissue is known. Elevated temperatures, or hyperthermia, may be harmful to the brain under normal conditions, and even more importantly, during periods of physical stress, such as illness or surgery. Conversely, lower body temperatures, or mild hypothermia, may offer some degree of neuroprotection. Moderate to severe hypothermia tends to be more detrimental to the body, particularly the cardiovascular system.

Targeted temperature management can be viewed in two different aspects. The first aspect of temperature management includes treating abnormal body temperatures, i.e., cooling the body under conditions of hyperthermia or warming the body under conditions of hypothermia. The second aspect of thermoregulation is an evolving treatment that employs techniques that physically control a patient's temperature to provide a physiological benefit, such as cooling a stroke patient to gain some degree of neuroprotection. By way of example, TTM systems may be utilized in early stroke therapy to reduce neurological damage incurred by stroke and head trauma patients. Additional applications include selective patient heating/cooling during surgical procedures such as cardiopulmonary bypass operations.

TTM systems circulate a fluid (e.g. water) through one or more thermal contact pads coupled to a patient to affect surface-to-surface thermal energy exchange with the patient. In general, TTM systems comprise a TTM fluid control module coupled to at least one contact pad via a fluid deliver line. One such TTM system is disclosed in U.S. Pat. No. 6,645,232, titled “Patient Temperature Control System with Fluid Pressure Maintenance” filed Oct. 11, 2001 and one such thermal contact pad and related system is disclosed in U.S. Pat. No. 6,197,045 titled “Cooling/heating Pad and System” filed Jan. 4, 1999, both of which are incorporated herein by reference in their entireties. As noted in the '045 patent, the ability to establish and maintain intimate pad-to-patient contact is of importance to fully realizing medical efficacies with TTM systems.

As these and other medical applications have evolved, the predictability, responsivity, flexibility, and portability of TTM systems have become more important. In some instances, a patient may need to be disconnected from the TTM system for a medical procedure and then reconnected after the medical procedure. In some instances, urgent disconnection may be required. As such, the reliability and ease of use of connections between a TTM module and the pads have become more important. For example, there is need for a clinician to be able to validate a secure connection between the fluid delivery line (FDL) and the thermal contact pad before TTM fluid flow is initiated. In some instances, it may be advantageous for a clinician to observe one or more parameters of the TTM fluid such as the temperature of the TTM fluid at the patient.

In some instances, the TTM control module may need to be located a distance away from the patient requiring a relatively long FDL (e.g., several feet in length). In such, instances heat transfer into or out of the TTM fluid may need to be identified and/or accounted for in the TTM therapy. As such an FDL which minimizes heat transfer to and away from the TTM fluid may be advantageous.

Disclosed herein are embodiments of devices and methods for the transportation of a TTM fluid to a thermal contact pad coupled to a patient.

### SUMMARY OF THE INVENTION

Briefly summarized, disclosed herein is a targeted temperature management (TTM) system. The TTM system may comprise a TTM module configured to provide a TTM fluid, a thermal pad configured to receive the TTM fluid from the TTM module to facilitate thermal energy transfer between the TTM fluid and a patient, and a multi-conduit fluid delivery line providing fluid communication between the TTM module and the thermal pad.

The TTM system may also comprise a connection system configured to provide a connectivity indication to a user. The connection system may comprise a first connector attached to the fluid delivery line, a corresponding second connector attached to the thermal pad, and a connectivity sensor configured to determine if the first connector is connected to the second connector. In some embodiments, the connectivity indication may be a confirming indication that the first connector is completely connected to the second connector and the connectivity indication may comprise an illuminating indicium. The connection system may also comprise one or more indicia indicating proper orientation of the first connector with respect to the second connector.

In some embodiments, the connection system may comprise a connection controller including controller logic coupled to the connectivity sensor. In some embodiments, the first connector comprises the connection controller. The connection system may include a display coupled to the connection controller, and the controller logic may be configured to render the connectivity indication on the display. In some embodiments, connection controller and the display may be disposed within a controller housing.

In some embodiments, the connection system includes a connector lock configured to selectively allow and prevent separation of the first connector from the second connector. The connection system may further include an actuator coupled to the connection controller and the connector lock, and the controller logic may be configured to selectively 1) activate the connector lock preventing separation of the first connector from the second connector, and 2) deactivate the connector lock allowing separation of the first connector from the second connector.

In some embodiments, the connection system may include a flow sensor configured to measure a TTM fluid flow rate. The flow sensor may be coupled to the connection controller, and the controller logic may be configured to render a TTM fluid flow rate indication on the display. The controller logic may also be configured to activate and/or deactivate the lock in accordance with a signal from the flow sensor.

In some embodiments, the connection system may further comprise a temperature sensor configured to measure a TTM fluid temperature. The temperature sensor may be coupled to the connection controller, and the controller logic may be configured to render a TTM fluid temperature indication on the display.

In some embodiments, the connection controller may be coupled to the TTM module, and the controller logic may provide the connectivity indication to the TTM module. The TTM module may comprise a TTM module console including TTM Module logic, and TTM module logic may be configured to selectively allow and prevent TTM fluid flow to the thermal pad in accordance with the connectivity indication. The controller logic may also be configured to provide one or more signals to the TTM module in accordance with signals received from the connection system, temperature sensor, and/or the flow sensor.

In some embodiments, the controller logic may be configured to render a visualization on the display indicating that separation of the first connector from the second connector is allowed in accordance with a signal received from the TTM module controller. The controller logic may also be configured to activate and/or deactivate the lock in accordance with a signal from the TTM module.

In some embodiments, the fluid delivery line comprises a first conduit and a second conduit, and the first conduit may be disposed within second conduit along a length of the fluid delivery line. A direction of fluid flow through the first conduit may be from the TTM module toward the thermal pad. The first conduit may be concentrically positioned within the second conduit, and the first conduit may be attached to the second conduit along the length of the fluid delivery line.

In some embodiments, the TTM system further includes a filter disposed in line with a TTM fluid flow path. The filter may comprise a porous wall disposed parallel to a flow direction of TTM fluid along the TTM fluid flow path. In some embodiments, the filter may be attached to the thermal pad, and in further embodiments, the filter may be disposed within a fluid containing layer of the thermal pad.

Disclosed herein is also a method of using a TTM system. The TTM system may include a TTM module configured to provide a TTM fluid, a thermal pad configured to receive the TTM fluid from the TTM module to facilitate thermal energy transfer between the TTM fluid and a patient, and a fluid delivery line (FDL) providing fluid communication between the TTM module and the thermal pad. The TTM system may also include a connection system and the connection system may include an FDL connector attached to the FDL and a pad connector attached to the thermal pad. The FDL connector may be configured to connect with the pad connector to establish fluid communication between the FDL and the thermal pad. The connection system may be configured to provide a visual indication to a user when the FDL connector is connected to the pad connector.

The method may further include connecting the FDL connector with the pad connector, observing the visual indication that the FDL connector is connected to the pad connector, and delivering TTM fluid from the TTM module to the thermal pad.

The connection system may further include a latching system configured to inhibit separation of the FDL connector from the pad connector, and the method may further include latching the FDL connector to the pad connector.

The connection system may further include a locking system configured to prevent separation of the FDL connector from the pad connector by a user, and the method may further include locking the FDL connector to the pad connector.

In some embodiments, the connection system may comprise a connection controller, and observing the visual indication may include observing the visual indication on a display of the controller. In some embodiments, the connection controller is coupled to the TTM module, and observing the visual indication comprises observing the visual indication on a display of the TTM module.

In some embodiments, the locking system may include a locking actuator coupled to the connection controller, and locking the FDL connector to the pad connector may include activating of the locking actuator by the connection controller. In some embodiments, the connection controller is coupled to the TTM module, and locking the FDL connector to the pad connector comprises activating the locking actuator in response to signal from the TTM module. In some embodiments, delivering TTM fluid is performed after activating the locking actuator.

Disclosed herein is also medical pad for facilitating thermal energy transfer between the TTM fluid and the patient. The pad may include a fluid containing layer and a fluid connector in fluid communication with the fluid containing layer. The fluid connector is configured to couple with a corresponding fluid line connector attached to the TTM fluid delivery line. The fluid connector may comprise a pad component of a connectivity system and the pad component may be configured to interact with a corresponding fluid line component of the connectivity system when the fluid connector is coupled with the corresponding fluid line connector. The connectivity system may be configured to provide an indication to a user that the fluid connector is coupled with the corresponding fluid line connector.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 illustrates a patient and a targeted temperature management (TTM) system for cooling or warming the patient, in accordance with some embodiments.

FIG. 2 illustrates a hydraulic schematic of the TTM system of FIG. 1, in accordance with some embodiments.

FIG. 3 illustrates a block diagram depicting various elements of a console of the TTM module of FIG. 1, in accordance with some embodiments.

FIG. 4 is a cross-sectional side view of the thermal contact pad of FIG. 1, in accordance with some embodiments.

FIG. 5A is a cross-sectional side view of a portion of the fluid delivery line of FIG. 1, in accordance with some embodiments.

FIGS. 5B-5F provide various cross-sectional end views of the fluid deliver line depicted in FIG. 5A, in accordance with some embodiments.

FIG. 6 is an illustration of the connection system of FIG. 1, in accordance with some embodiments.

FIG. 7 illustrates a block diagram of a connection controller of the connection system of FIG. 6, in accordance with some embodiments.

FIG. 8A provides an exploded perspective view of a TTM fluid filter, in accordance with some embodiments.

FIG. 8B provides a cross-sectional side view of the filter of FIG. 8A, in accordance with some embodiments.

FIG. 8C provides a side cross-sectional view of the thermal contact pad of FIG. 1 incorporating the filter of FIG. 8A, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. The words “including,” “has,” and “having,” as used herein, including the claims, shall have the same meaning as the word “comprising.” Furthermore, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The phrases “connected to” and “coupled to” refer to any form of interaction between two or more entities, including mechanical, electrical, magnetic, electromagnetic, fluid, signal, communicative (including wireless), and thermal interaction. Two components may be connected or coupled to each other even though they are not in direct contact with each other. For example, two components may be coupled to each other through an intermediate component.

Unless defined otherwise, all technical and scientific terms used herein have the same meaning as commonly understood by those of ordinary skill in the art.

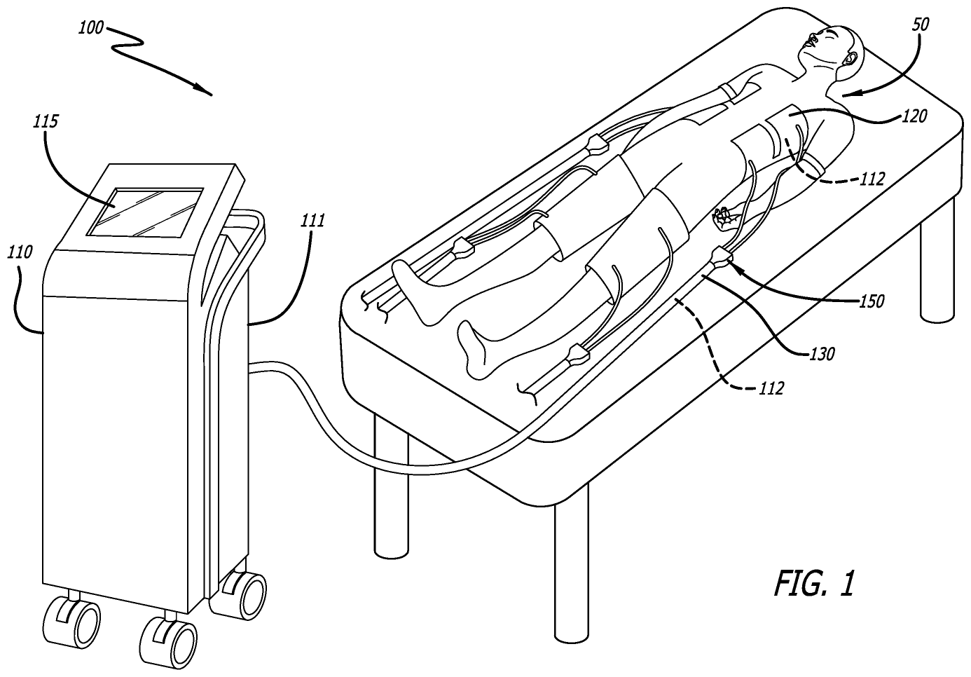

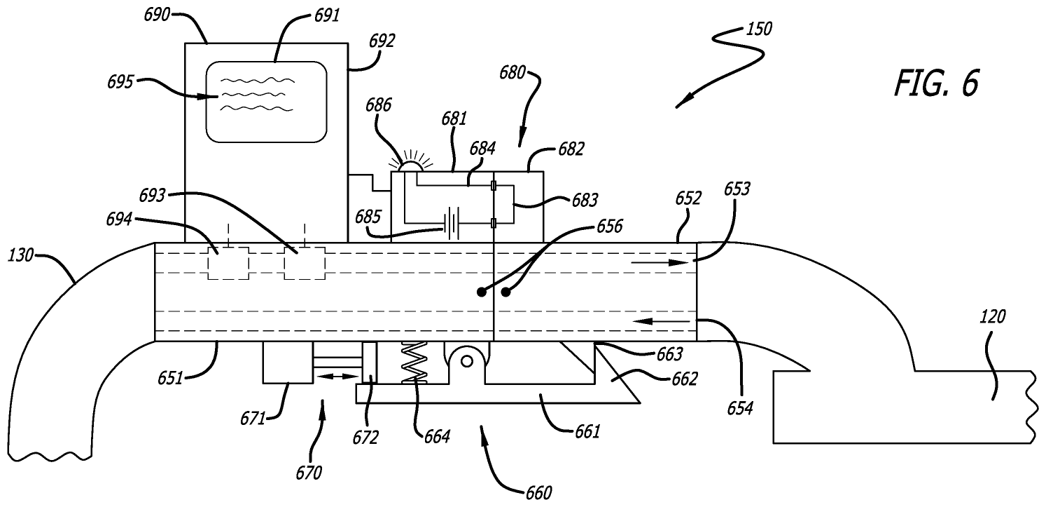

FIG. 1 illustrates a targeted temperature management (TTM) system 100 connected to a patient 50 for administering targeted temperature management therapy to the patient 50 which may include a cooling and/or warming of the patient 50, in accordance with some embodiments. The TTM system 100 comprises a TTM module 110 including a graphical user interface (GUI) 115 enclosed within a module housing 111. The TTM system 100 includes a fluid deliver line (FDL) 130 extending from the TTM module 110 to a thermal contact pad (pad) 120 to provide for flow of TTM fluid 112 between the TTM module 110 and the pad 120. The TTM system 100 may include a connection system 150 to couple the FDL 130 to the pad 120. Additional detail on the connection system 150 is illustrated in at least FIG. 6 and described below.

The TTM system 100 may include 1, 2, 3, 4 or more pads 120 and the TTM system 100 may include 1, 2, 3, 4 or more fluid delivery lines 130. In use, the TTM module 110 prepares the TTM fluid 112 for delivery to the pad 120 by heating or cooling the TTM fluid 112 to a defined temperature in accordance with a prescribed TTM therapy. The TTM module 110 circulates the TTM fluid 112 within the pad 120 to facilitate thermal energy exchange with the patient 50. During the TTM therapy, the TTM module 110 may continually control the temperature of the TTM fluid 112 toward a target TTM temperature. In some instances, the target TTM temperature may change during the TTM therapy.

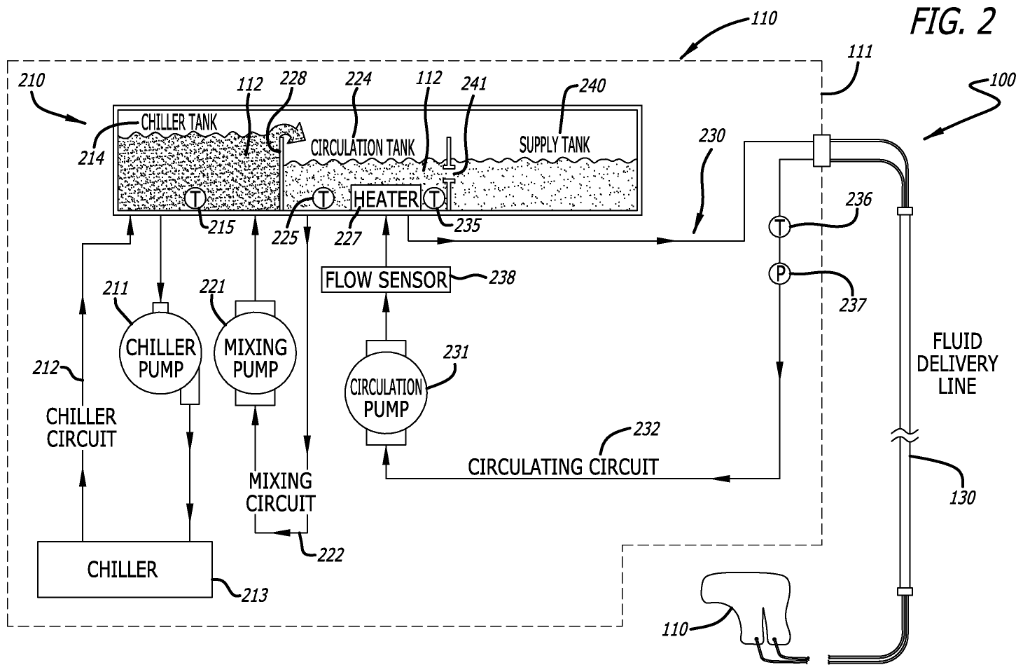

FIG. 2 illustrates a hydraulic schematic of the TTM system 100. The FDL 130 and the pad 120 are disposed external to the housing 111 of the TTM module 110. The TTM module includes various fluid sensors and fluid control devices to prepare and circulate the TTM fluid 112. The fluid subsystems of the TTM module may include a temperature control subsystem 210 and a circulation subsystem 230.

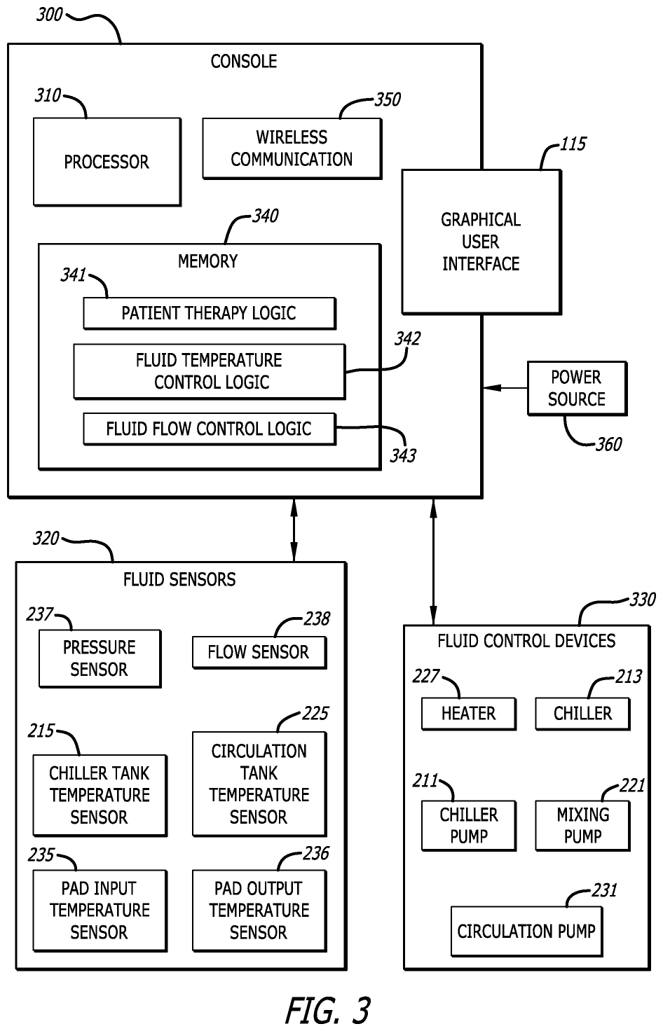

The temperature control subsystem 210 may include a chiller pump 211 to pump (recirculate) TTM fluid 112 through a chiller circuit chiller 212 that includes a chiller 213 and a chiller tank 214. A temperature sensor 215 within the chiller tank 214 is configured to measure a temperature of the TTM fluid 112 within the chiller tank 214. The chiller 213 may be controlled by a temperature control logic (see FIG. 3) as further described below to establish a desired temperature of the TTM fluid 112 within chiller tank 214. In some instances, the temperature of the TTM fluid 112 within the chiller tank 214 may be less than the target temperature for the TTM therapy.

The temperature control subsystem 210 may include may further include a mixing pump 221 to pump TTM fluid 112 through a mixing circuit 222 that includes the chiller tank 214, a circulation tank 224, and a dam 228 disposed between the chiller tank 214 and circulation tank 224. The TTM fluid 112, when pumped by the mixing pump 221, enters the chiller tank 214 and mixes with the TTM fluid 112 within the chiller tank 214. The mixed TTM fluid 112 within the chiller tank 214 flows over the dam 228 and into the circulation tank 224. In other words, the mixing circuit 222 mixes the TTM fluid 112 within chiller tank 214 with the TTM fluid 112 within circulation tank 224 to cool the TTM fluid 112 within the circulation tank 224. A temperature sensor 225 within the circulation tank 224 measures the temperature of the TTM fluid 112 within the circulation tank 224. The temperature control logic may control the mixing pump 221 in accordance with temperature data from the temperature sensor 225 within the circulation tank 224.

The circulation tank 224 includes a heater 227 to increase to the temperature of the TTM fluid 112 within the circulation tank 224, and the heater 227 may be controlled by the temperature control logic. In summary, the temperature control logic when executed by the processor (see FIG. 3) may 1) receive temperature data from the temperature sensor 215 within the chiller tank and the temperature sensor 225 within the circulation tank 224 and 2) control the operation of the chiller 213, the chiller pump 211, the heater 227, and mixing pump 222 to establish and maintain the temperature of the TTM fluid 112 within the circulation tank 224 at the target temperature for the TTM therapy.

The circulation subsystem 230 comprises a circulation pump 213 to pull TTM fluid 112 from the circulation tank 224 and through a circulating circuit 232 that includes the fluid delivery line 120 and the pad 120 located upstream of the circulation pump 213. The circulating circuit 232 also includes a pressure sensor 237 to represent a pressure of the TTM fluid 112 within the pad 120. The circulating circuit 232 also includes a temperature sensor 235 within the circulation tank 224 to represent the temperature of the TTM fluid 112 entering the pad 120 and a temperature sensor 236 to represent the temperature of the TTM fluid exiting the pad 120. A flow meter 238 is disposed downstream of the circulation pump 213 to measure the flow rate of TTM fluid 112 through the circulating circuit 232 before the TTM fluid 112 re-enters that the circulation tank 224.

In use, the circulation tank 224, which may be vented to atmosphere, is located below (i.e., at a lower elevation) the pad 120 so that a pressure within the pad 120 is less than atmospheric pressure (i.e., negative) when fluid flow through the circulating circuit 232 is stopped. The pad 120 is also placed upstream of the circulation pump 231 to further establish a negative pressure within the pad 120 when the circulation pump 213 is operating. The fluid flow control logic (see FIG. 3) may control the operation of the circulation pump 213 to establish and maintain a desired negative pressure within the pad 120. A supply tank 240 provides TTM fluid 112 to the circulation tank 224 via a port 241 to maintain a defined volume of TTM fluid 112 within the circulation tank 224.

FIG. 3 illustrates a block diagram depicting various elements of the TTM module 110 of FIG. 1, in accordance with some embodiments. The TTM module includes a console 300 including a processor 310 and memory 340 including non-transitory, computer-readable medium. Logic modules stored in the memory 340 include patient therapy logic 341, fluid temperature control logic 342, and fluid flow control logic 343. The logic modules when executed by the processor 310 define the operations and functionality of the TTM Module 110.

Illustrated in the block diagram of FIG. 3 are fluid sensors 320 as described above in relation to FIG. 2. Each of the fluid sensors 320 are coupled to the console 300 so that data from the fluid sensors 320 may be utilized in the performance of TTM module operations. Fluid control devices 330 are also illustrated in FIG. 3 as coupled to the console 300. As such, logic modules may control the operation of the fluid control devices 330 as further described below.

The patient therapy logic 341 may receive input from the clinician via the GUI 115 to establish operating parameters in accordance with a prescribed TTM therapy. Operating parameters may include a target temperature for the TTM fluid 112 which may comprise a time-based target temperature profile. In some embodiments, the fluid temperature control logic 342 may define other fluid temperatures of the TTM fluid 112 within the TTM module 110, such a target temperature for the TTM fluid 112 within the chiller tank 214, for example.

The fluid temperature control logic 342 may perform operations to establish and maintain a temperature of the TTM fluid 112 delivered to the pad 120 in accordance with a predefined target temperature profile. One temperature control operation may include chilling the TTM fluid 112 within the chiller tank 214. The fluid temperature control logic 342 may utilize temperature data from the chiller tank temperature sensor 215 to control the operation of the chiller 213 to establish and maintain a temperature of the TTM fluid 112 within the chiller tank 214.

Another temperature control operation may include cooling the TTM fluid 112 within the circulation tank 224. The fluid temperature control logic 342 may utilize temperature data from the circulation tank temperature sensor 225 to control the operation of the mixing pump 221 to decrease the temperature of the TTM fluid 112 within the circulation tank 224.

Still another temperature control operation may include warming the TTM fluid 112 within the circulation tank 224. The fluid temperature control logic 342 may utilize temperature data from the circulation tank temperature sensor 225 to control the operation of the heater 227 to increase the temperature of the TTM fluid 112 within the circulation tank 224.

The fluid flow control logic 343 may control the operation of the circulation pump 231. As a thermal energy exchange rate is at least partially defined by the flow rate of the TTM fluid 112 through the pad 120, the fluid flow control logic 343 may, in some embodiments, control the operation of the circulation pump 231 in accordance with a defined thermal energy exchange rate for the TTM therapy.

The console 300 may comprise wireless communication capability 350 to facilitate wireless communication with the connection system 150 and/or other external devices. A power source 360 provides electrical power to the console 300.

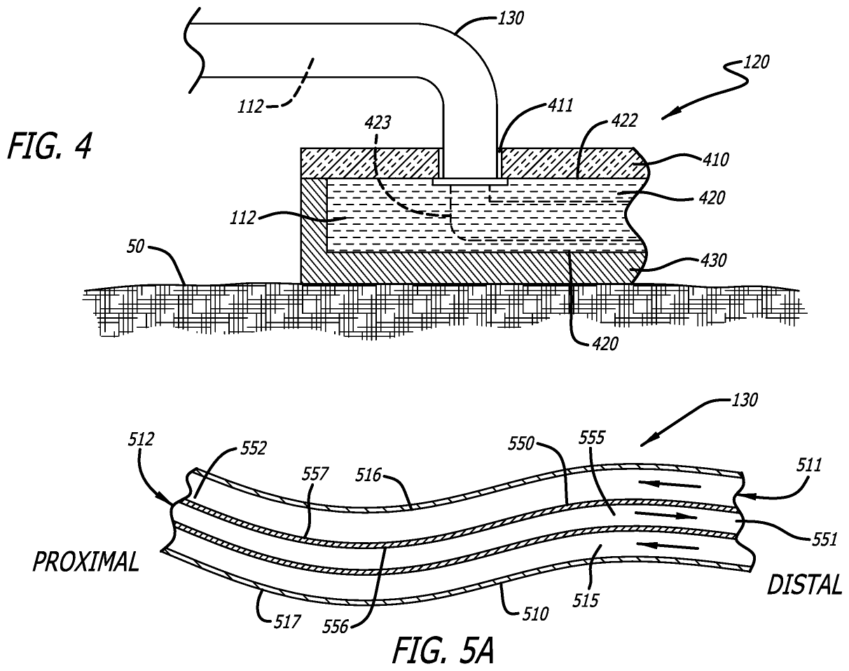

FIG. 4 shows a cross-sectional side view of an inlet or an outlet of the thermal contact pad 120 of FIG. 1 in contact with the patient 50, in accordance with some embodiments. The pad 120 may comprise multiple layers to provide multiple functions of the pad 120. A fluid containing layer 420 is fluidly coupled to the FDL 130 to facilitate circulation of the TTM fluid 112 within the fluid containing layer 420. The fluid containing layer 420 having TTM fluid 112 circulating therein defines a heat sink or a heat source for the patient 50 in accordance with a temperature of the TTM fluid 112.

The pad 120 may include a thermal conduction layer 430 disposed between the fluid containing layer 420 and the patient 50. The thermal conduction layer 430 is configured to facilitation thermal energy transfer between the fluid containing layer 420 and the patient 50. The thermal conduction layer 430 may be attached to the thermal conduction layer 430 along a bottom surface 421 of the thermal conduction layer 430. The thermal conduction layer 430 may be conformable to provide for intimate contact with the patient 50. In other words, thermal conduction layer 430 may conform to a contour of the patient 50 to inhibit space or air pockets between the thermal conduction layer 430 and the patient 50.

The pad 120 may include an insulation layer 410 disposed on the top side of the fluid containing layer 420. The insulation layer 410 is configured to inhibit thermal energy transfer between the fluid containing layer 420 and the environment. The insulation layer 410 may be attached to the fluid containing layer 420 along a top surface 422 of the fluid containing layer 420. In some embodiments, the insulation layer 410 may comprise one or more openings 411 extending through the insulation layer 410 to provide for coupling of the FDL 130 with the fluid containing layer 420.

In some embodiments, the opening 411 illustrates an inlet port to which the FDL 130 couples such that the TTM fluid 112 may enter into the fluid containing layer 420 and flow freely in a direction as dictated by the negative pressure within the pad 120 resulting from operation of the circulation pump 213. However, in other embodiments, the fluid containing layer 420 may include one or more internal flow paths (illustrated via dashed lines) such that the TTM fluid 112 may flow through the internal flow path(s) in a controlled manner in the as dictated by the negative pressure resulting from operation of the circulation pump 213. In some embodiments, e.g., in which FIG. 4 illustrates an inlet port, the TTM fluid 112 exits the pad 120, and specifically the fluid containing layer 420 via an outlet port (not shown) that may resemble the configuration of the as shown in FIG. 4.

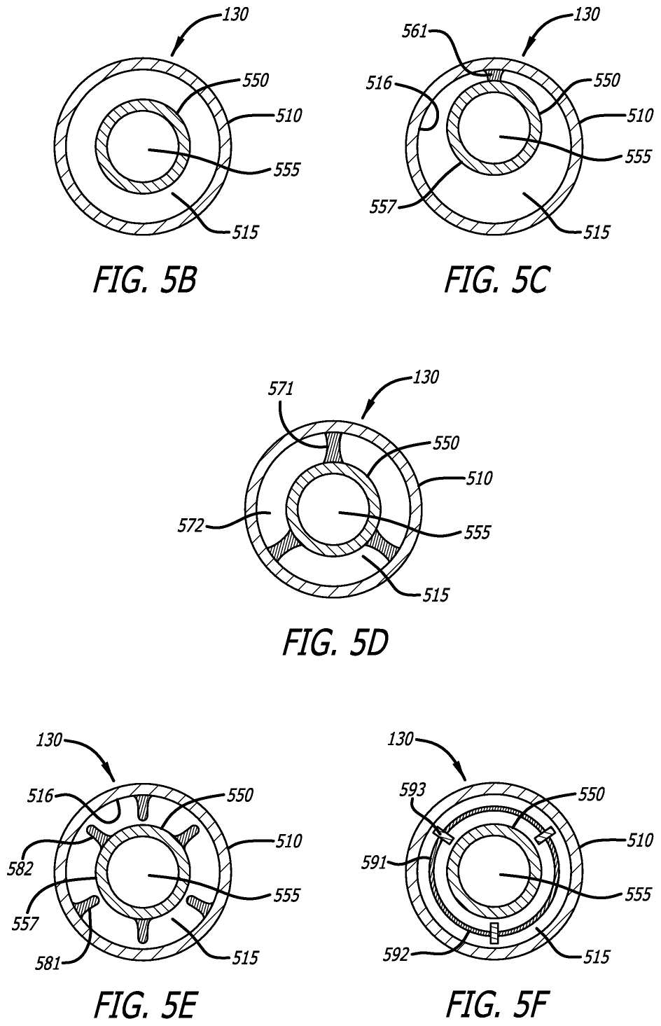

FIG. 5A shows a cross-sectional side view of the FDL 130. The FDL 130 comprises an outer conduit 510 extending between a distal end 511 and proximal end 512. The FDL 130 also comprises an inner conduit 550 extending between a distal end 551 and proximal end 552 and defining an inner flow path 555. An outer flow path 515 is defined by the annular area between the inside surface 516 of the outer conduit 510 and the outside surface 557 of the inner conduit 550. The inner conduit 510 and the outer conduit 550 may have the same length. In some embodiments, one of the inner conduit 510 or the outer conduit 550 may be longer than the other.

The outer conduit 510 may be formed of a plastic material via an extrusion process. The outer conduit 510 comprise one or more reinforcements (not shown) extending the length of the outer conduit 510. The reinforcements may inhibit crushing and/or kinking of the outer conduit 510 during use. The reinforcements may also inhibit collapsing of the conduit 510 during use under negative pressure. The outer conduit 510 may comprise indicia (not shown) on an outer surface 517 and the outer surface may be smooth or textured. The outer conduit 510 may comprise a round cross-section having an inside diameter between x and Y mm. In some embodiments, the outer conduit may have a diameter within the range of 1-3 inches. In one illustrative embodiment, the outer conduit may be approximately two inches in diameter.

The outer conduit 510 may be configured to minimize heat transfer between the TTM fluid 112 and the environment. In some embodiments, the structure of the outer conduit 510 may comprise thermal insulative properties, such as a foam structure, for example. To further inhibit heat transfer, the inside surface 516 may be smooth to facilitate fluid flow in the laminar region. Fluid flow in the laminar region may minimize convective heat transfer of the TTM fluid 112 to the inside surface of the outer conduit 510. In some embodiments, the inside surface 516 may comprise an antimicrobial coating (not shown) to inhibit bacterial growth.

Similar to the outer conduit 510, the inner conduit 550 may be formed of a plastic material via the extrusion process. The inner conduit 550 may comprise a structure to inhibit collapsing of the conduit 510 during use under negative pressure. The inner conduit 550 may be configured to minimize heat transfer between the TTM fluid 112 flowing within the inner conduit 112 and the TTM fluid 112 flowing along the outside surface 557. In some embodiments, the structure of the inner conduit 550 may comprise thermal insulative properties, such as multiple longitudinal extruded lumens (not shown) within the wall of the inner conduit 550, for example. To further inhibit heat transfer, the inside surface 556 may be smooth to facilitate fluid flow in the laminar region within the inner flow path 555. Fluid flow in the laminar region may minimize convective heat transfer of the TTM fluid 112 to the inside surface of the inner conduit 550. To further inhibit heat transfer, the outside surface 557 may be smooth to facilitate fluid flow in the laminar region within the outer flow path 515. Fluid flow in the laminar region along the outer flow path may minimize convective heat transfer of the TTM fluid 112 to the outside surface 557 of the inner conduit 550. In some embodiments, the inside surface 556 and the outside surface 557 may comprise an antimicrobial coating (not shown) to inhibit bacterial growth.

FIGS. 5B-5F show cross-sectional end views of different embodiments of the FDL 130. FIG. 5B shows an embodiment of the FDL 130 where the inner conduit 550 is not attached to the outer conduit 510. Allowing the inner conduit 550 to float relative to the outer conduit 510 may provide for greater flexibility of the FDL 130.

FIG. 5C shows an embodiment of the FDL where the inner conduit 550 is attached to the outer conduit 510 via longitudinal rib 561. The embodiment of FIG. 5C may comprise a single extrusion. In this embodiment, a longitudinal axis of the inner conduit 550 is offset from a longitudinal axis of the outer conduit 510. The embodiment of FIG. 5C may comprise more than one rib 561. Offsetting the inner conduit 550 relative to the outer conduit 510 may provide for less heat transfer between the TTM fluid 112 flowing within the inner flow path 555 and the TTM fluid 112 flowing along the outer flow path 515.

FIG. 5D shows an embodiment of the FDL 130 similar to the embodiment of FIG. 5C. In this embodiment, the inner conduit 550 is concentrically positioned within the outer conduit 510. The inner conduit 550 is attached to the outer conduit 510 via one or more longitudinal ribs 571. The embodiment of FIG. 5D may comprise a single extrusion. In the illustrated embodiment, the outer flow path 515 is divided into three flow paths 572. The number of longitudinal ribs 571 may be 1, 2, 3, 4 or more.

FIG. 5E shows an embodiment where (1) the outer conduit 510 comprises one or more ribs 581 protruding inward from the inside surface 516 and/or (2) the inner conduit 550 comprises one or more ribs 582 protruding outward from the outside surface 557. The ribs 581, 582 may be configured to constrain the inner conduit 550 to be concentric with the outer conduit 510. In this embodiment, inner conduit 550 and the outer conduit 510 may comprise separate extrusions.

FIG. 5F shows an embodiment similar to the embodiment of FIG. 5B in that each of the inner conduit 550 and the outer conduit 510 are separately extruded tubes. In this embodiment a separating structure 591 is disposed between the inner conduit 550 and the outer conduit 510 to constrain the inner conduit 550 to be concentric with the outer conduit 510. The separating structure 591 may comprise a hoop portion 592 coupled to multiple longitudinal rib portions 593. The separating structure 591 may formed of an extrusion.

FIG. 6 provides an illustration of the connection system 150 of FIG. 1 including a fluid line connector 651 and a corresponding pad connector 652. FIG. 6 further illustrates a latching system 660, a locking system 670, and a connectivity system 680. In some embodiments, the connection system 150 includes a connection controller 690 and which may include a display 691. In some embodiments, the display 691 may be disposed within a connection controller housing 692.

The fluid line connector 651 is fluidly attached to the FDL 130 and the pad connector 652 is attached to the pad 120. The fluid line connector 651 and pad connector 652 establish fluid communication between the FDL 130 and the pad 120. In some embodiments, the attachment of the fluid line connector 651 with the FDL 130 may provide for rotation of the fluid line connector 651 with respect to the FDL 130. Similarly, the attachment of the pad connector 652 with the pad 120 may provide for rotation of the pad connector 652 with respect to the pad 120.

The connectors 651, 652 are configured to facilitate passage of the TTM fluid 112 between the FDL 130 and the pad 120 when the fluid line connector 651 is coupled to the pad connector 652. The connectors 651, 652, when connected together, comprise a first lumen 653 extending between the FDL 130 and the pad 120 to facilitate passage of the TTM fluid 112 from the FDL 130 to the pad 120. In some embodiments, the connectors 651, 652, when connected together, may comprise a second lumen 654 extending between the FDL 130 and the pad 120 to facilitate passage of the TTM fluid 112 from the pad 120 to the FDL 130. The lumens 653, 654 may be arranged in a side by side configuration or in a coaxial configuration. The connectors 651, 652 are also configured to provide a sealing connection to prevent leakage of the TTM fluid 112 out of the lumens 653, 654. In some embodiments, the second lumen 654 may be omitted.

The connectors 651, 652 may be configured to provide for selective connection and disconnection via displacement of connectors 651, 652 with respect to each other. The displacement, may be a longitudinal displacement, a lateral displacement, or a rotational displacement.

The connection system may include indicia 656 on an outside surface of the connectors 651, 652. Complementary portions of the indicia 656 may be included on each of the connectors 651, 652. The indicia 656 may include alignment markings to indicate to a user that the connectors 651, 652 are in proper alignment for connection, such as rotational alignment, for example.

The connection system 150 may provide a detent (not shown) that inhibits disconnection of the connectors 651, 652 under normal use or in the absence of a deliberate separating force applied by the user. The detent may provide audible and/or tactile feedback to the user that the connectors 651, 652 are completely connective to each other.

In some embodiments, the connection system 150 may include a latching system 660 such as the exemplary latching system 660 illustrated in FIG. 6. In the illustrated embodiment, the latching system 660 comprises a biased lever 661 pivotably attached to the fluid line connector 651. The lever 661 includes a hook 662 configured to engage a corresponding ledge 663 coupled to the pad connector 652. A spring 664 biases the lever 661 in a latched position. Operating the lever 661 comprises pivoting the lever 661 to disengage the hook 664 from the ledge 663. As may be appreciated by one of ordinary skill, the latching system 660 may comprise any latching mechanism that 1) inhibits separation of the connectors 651, 652 in the absence a deliberate action by the user and 2) allows separation of the connectors 651, 652 in response to a deliberate action by the user. Deliberate actions by the user may include operating a lever (such as the lever 661 illustrated in FIG. 6), pushing a button, rotating one of the connectors 651, 652 relative to the other connector, pushing the connecters 651, 652 toward each other, longitudinally displacing a collar, rotating a collar, or any other deliberate action as may be appreciated by one of ordinary skill. In some embodiments, the latching system 660 may provide audible and/or tactile feedback to the user that the connectors 651, 652 are latched to each other.

In some embodiments, the connection system 150 may include a locking system 670 to selectively prevent and allow a user from separating the connectors 651, 652. The locking system 670 may comprise any mechanism that can selectively enable and disable operation of the latching system 660. The locking system 670 may include an actuator 671. The actuator 671 may be an electro-mechanical actuator, such as a solenoid, for example. In the exemplary locking system 670, the actuator 671 selectively displaces a plunger 672 to engage with and disengage from the lever 661 of the latching system 660. As illustrated, when the plunger 672 is extended, operation of the lever 661 is prevented. In contrast, when the plunger 672 is retracted, operation of the lever 672 is enabled to allow a user to deactivate the latching system 660. The actuator 671 may be configured to enable and/or disable deactivation of the latching system 660 in response to a signal from the connection controller 690 as further described below. In some embodiments, the locking system 670 and the latching system 660 may be combined so that the actuator 671 may directly activate and/or deactivate the latching system 660 in response to a signal from the connection controller 690. In some embodiments, the locking system 670 may be biased toward a default configuration wherein operation of the latching system 660 is enabled (i.e., unlocked).

In some embodiments, the connection system 150 may include a connectivity system 680. The connectivity system 680 may be configured to determine if the connectors 651, 652 are fully connected together. In some embodiments, the connectivity system 680 may be configured to provide a signal to the connection controller 690 as to the connection status of the connectors 651, 652.

The connectivity system 680 may comprise complementary sensor components that interact with each other. The connectivity system 680 may comprise a first sensor component 685 attached to the fluid line connector 651 and a complementary second sensor component 686 attached to the pad connector 652. The sensor system 680 may be configured to provide a “connected” signal if and only if the first sensor component 685 is interactively coupled to the second sensor component 686. In the exemplary illustrated embodiment, the first sensor component 685 comprises an open electrical circuit 684 including a power source 685 and an illumination device 686. The second sensor component 686 comprises an electrical conductor 683. The first sensor component 685 and the second sensor component 686 are arranged so that the upon complete connection of the fluid line connector 651 with the pad connector 652, the electrical conductor of the second sensor component 686 completes the circuit of the first sensor component 685 which in turn generates a “connected” indication such as powering the illumination device 686.

In some embodiments, the first sensor component 685 may be an active component (i.e. a component requiring electrical power for operation) and the second sensor component 686 may be a passive component. As may be appreciated by one of ordinary skill, the sensor system 680 may comprise any suitable sensor technology capable of providing a signal when two physical components (i.e. the fluid line connector 651 and the pad connector 652) are disposed adjacent to or in close proximity to one another. Exemplary sensor technologies include: electrical conductance, capacitance, or inductance; optical interruption or reflection; hall effect sensors and magnetism; proximity; and the like.

In some embodiments, the connection system 150 may include a temperature sensor 693 configured to measure a temperature of the TTM fluid 112 within the first lumen 653. As discussed above, TTM fluid 112 within the first lumen flows toward the pad 120. Measuring the temperature of the TTM fluid 112 at the connector may provide a more accurate temperature measurement of the TTM fluid 112 within the pad 120 than a temperature measurement at the TTM module 110 because the temperature of the TTM fluid may be affected during transportation through the FDL 130. The temperature sensor 691 may be coupled to the connection controller 690 and provide a signal thereto. In some embodiments, the connection system 150 may also comprise a second temperature sensor (not shown) configured to measure a temperature of the TTM fluid 112 within the second lumen 654.

In some embodiments, the connection system 150 may include a flow sensor 694 configured to measure a flow rate of the TTM fluid 112 through the first lumen 653. As discussed above, the TTM fluid 112 within the first lumen flows toward the pad 120. Measuring the flow rate of the TTM fluid 112 at the fluid line connector 651 may be indicative of a blockage of flow from the TTM module 110 to the pad 120 and/or a leak of air into the first lumen 653. The flow sensor 694 may be coupled to the connection controller 690 and provide a signal thereto. In some embodiments, the connection system 150 may also comprise a second flow sensor (not shown) configured to measure a flow of the TTM fluid 112 within the second lumen 654.

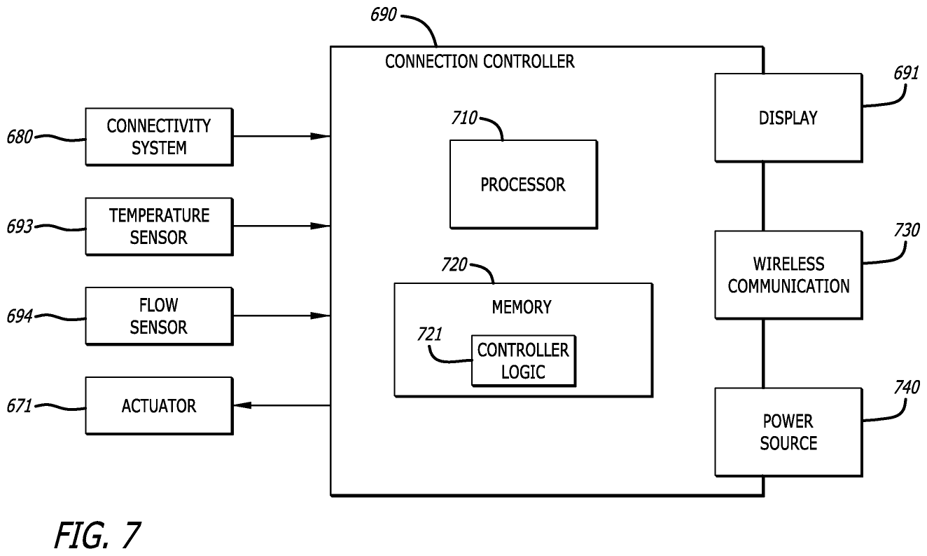

FIG. 7 illustrates a block diagram of the connection controller 690. The connection controller incudes a processor 710 and memory 710 which may be a non-transitory, computer-readable medium. The memory 710 includes controller logic 721 stored therein. A display 691 may be coupled to the connection controller 690. The connection controller 690 may be coupled to a power source 740 or the connection controller 690 may be powered internally with a battery. In some embodiments, the power source 740 may comprise a generator operatively coupled to the TTM fluid 112 so that electrical power is provide to the controller 690 when TTM fluid 112 flows through the first lumen 653. The connection controller 690 may comprise wireless communication capability 730 to facilitate wireless communication with the TTM Module 110 and/or other external devices. The connection controller 690 may be coupled to the connectivity system 680, the temperature sensor 693, and the flow sensor 694. The controller logic 721 may be configured receive data from the connectivity system 680, the temperature sensor 693, and the flow sensor 694. The connection controller 690 may also be coupled to actuator 671 and the controller logic 721 may be configured to activate and/or deactivate the actuator 671 when the controller logic 721 is executed by the processor 710.

The control logic 721 may be configured to activate the actuator and thereby dispose the locking system 670 into a locked configuration upon receipt of various signals. The control logic 721 may be configured to activate the actuator 671 when a signal from the connection system 680 indicates that the connectors 651,652 are completely coupled together. The control logic 721 may be configured to activate the actuator 671 upon a signal from the flow sensor 694 indicating flow of TTM fluid 112 toward the pad 120. The control logic 721 may be configured to activate the actuator 671 upon a signal from the TTM module 110 indicating that flow of TTM fluid 112 toward the pad 120 is initiated. The control logic 721 may be configured to activate the actuator 671 upon a signal from the TTM module 110 that corresponds to a user input.

In similar fashion, the control logic 721 may be configured to deactivate the actuator and thereby dispose the locking system 670 into an unlocked configuration upon receipt of various signals. The control logic 721 may be configured to deactivate the actuator 671 upon a signal from the flow sensor 694 indicating no flow of TTM fluid 112 toward the pad 120. The control logic 721 may be configured to deactivate the actuator 671 upon a signal from the TTM module 110 indicating that flow of TTM fluid 112 toward the pad 120 is stopped. The control logic 721 may be configured to deactivate the actuator 671 upon a signal from the TTM module 110 that corresponds to a user input. The control logic 721 may be configured to deactivate the actuator 671 upon a signal from the TTM module 110 indicating loss of power to the TTM module 110. The control logic 721 may be configured to deactivate the actuator 671 upon a signal from the TTM module 110 indicating that the TTM module 110 is operating outside of intended operating parameters. The control logic 721 may be configured to deactivate the actuator 671 upon a signal from the temperature sensor 693 indicating that the temperature of the TTM fluid 112 exceeds a defined temperature range. The control logic 721 may be configured to deactivate the actuator 671 upon a signal from the TTM module 110 indicating that a TTM therapy is complete.

In some embodiments, the control logic 721 may be configured to render indicia 695 on the display 691. The indicia 695 may include indications pertaining to the connection status of the connection system 150. The indicia 695 may indicate that the connectors 651, 652 are completely connected together and/or that the connectors 651, 652 are not completely connected together. The indicia 695 may indicate if the locking system is activated and/or not activated. The indicia 695 may indicate a temperature of the TTM fluid 112 and/or if the temperature of the TTM fluid 112 exceeds a defined range. The indicia 695 may indicate a flow rate of the TTM fluid 112 and/or if the flow rate of the TTM fluid 112 exceeds a defined range. The indicia 695 may include information from the TTM module 110 pertaining to the TTM therapy, such as the remaining therapy time, for example. The indicia 695 may include information from the TTM module 110 pertaining to operating parameters of the TTM module 110.

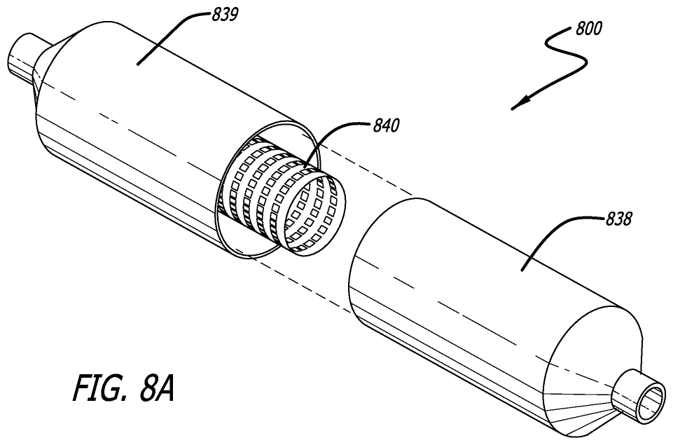

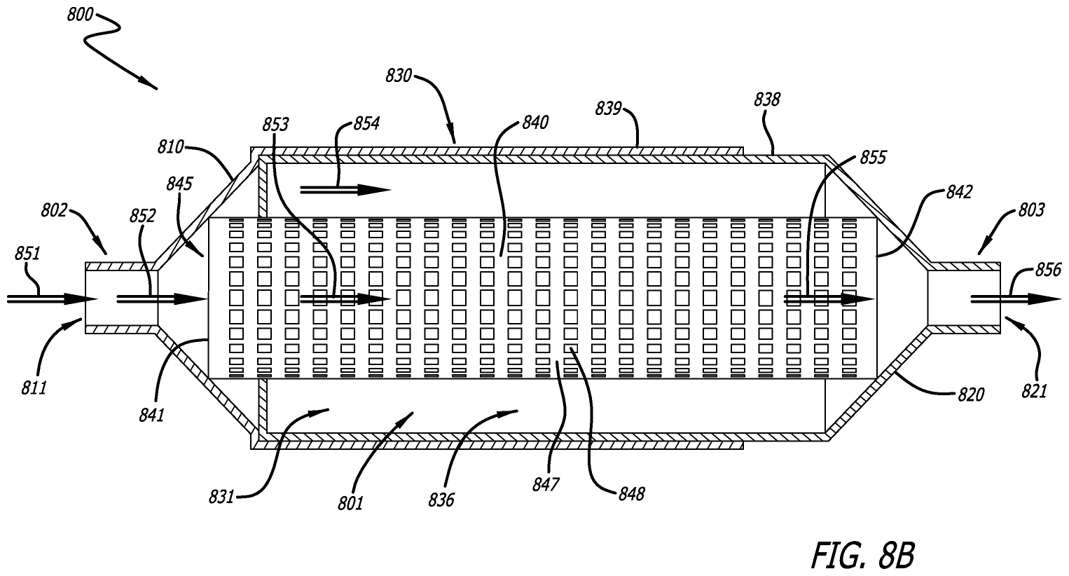

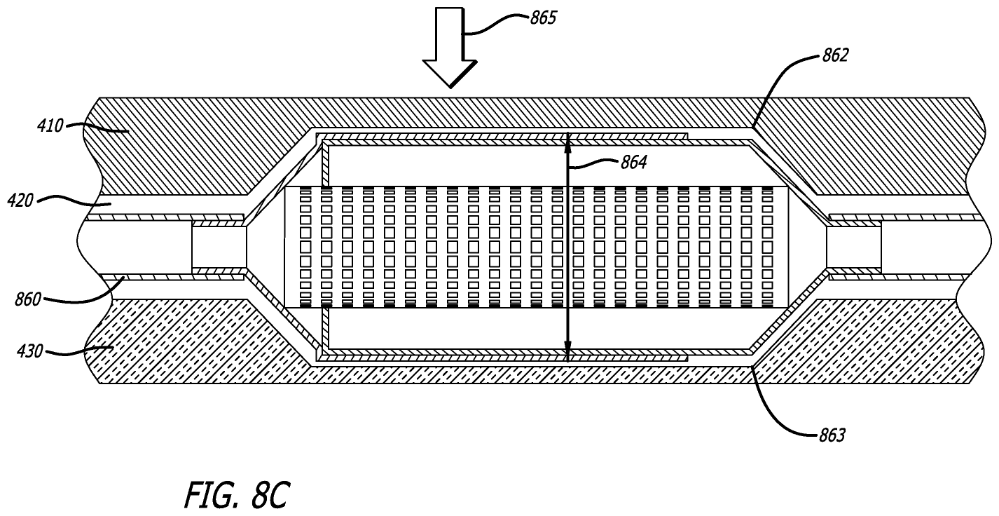

The TTM system 100 may include a filter 800 as shown in FIGS. 8A-8B. The filter 800 may be disposed in line with a TTM fluid flow path of the TTM system 100 so that the circulating TTM fluid 112 flows through the filter 800. The filter 800 may be configured to remove (i.e., filter out) material/particles having a size of 0.2 microns or larger from the TTM fluid 112 without causing a flow restriction of the TTM fluid 112.

The filter 800 comprises a longitudinal shape having a flow path 801 extending from a first end 802 to a second end 803. The filter 800 comprises a diffuser 810 adjacent the first end 802, a nozzle adjacent 820 the second end 803, and a body 830 extending between the diffuser 810 and the nozzle 820. Along the diffuser 810, a cross-sectional flow area of the filter 800 expands from an inlet flow area 811 to a body flow area 831 and along the nozzle 820, the cross-sectional flow area of the filter 800 contracts from the body flow area 831 to an outlet flow area 821. In some embodiments, the inlet flow area 811 and the outlet flow area 821 may be substantially equal.

In some embodiments, the body flow area 831 may be constant along the body 830. In other embodiments, the body flow area 831 may vary along a length of the body 830 such that the body flow area 831 is greater or less along middle portion of the body 830 than at the ends of the body 830. In some embodiments, the body flow area 831 may be circular.

The filter 800 comprises an inner tube 840 disposed within the body 830 extending along the length of body 830. The inner tube 840 may be coupled to the diffuser 810 at a first inner tube end 841 so that TTM fluid 112 entering the filter 800 at the first end 802 also enters the inner tube 840 at the first inner tube end 841. The inner tube 840 may be coupled to the nozzle 820 at a second inner tube end 842 so that TTM fluid 112 exiting the filter 800 at the second end 803 also exits the inner tube 840 at the second inner tube end 842.

The inner tube 840 comprises an inner tube flow area 845 extending the length of the inner tube 840. The inner tube flow area 845 may be greater than the inlet flow area 811 and/or the outlet flow area 821. The inner tube flow area 845 may be constant along the length of the inner tube 840. In some embodiments, the inner tube flow area 845 may vary along the length of the inner tube 840. In some embodiments, the inner tube 840 may comprise a circular cross section. The inner tube 840 and the body 830 may be configured so that the body flow area 831 comprises a combination of the inner tube flow area 845 and an annular flow area 836.

The inner tube 840 comprises a porous a circumferential wall 847. The porous wall 847 may be configured so that TTM fluid 112 may flow through the porous wall 847, i.e., through the pores 848 of the porous wall 847. Consequently, TTM fluid 112 may flow through the porous wall 847 from the inner tube flow area 845 to the annular flow area 836 and from the annular flow area 836 into the inner tube flow area 845.

In various embodiments, the pores 848 may taking differing shapes such as, e.g., louver, counter sunk, round, oblong, etc. Further, in some embodiments, the pores 848 may have differing shapes within a particular filter 800, which may improve filtration by providing varying apertures through which sediment may fall.

In use, the longitudinal velocity of the TTM fluid 112 may change along the length of the filter 800. As the volumetric TTM fluid 112 flow through the filter is constant, the longitudinal velocity of the TTM fluid 112 may be at least partially defined by the flow areas of the filter 800 as described below. The TTM fluid 112 may enter the filter 800 at a first longitudinal velocity 851 and decrease along the diffuser so that the TTM fluid 112 enters the inner tube at a second velocity 852 less than the first longitudinal velocity 851. At this point, a portion of the TTM fluid 112 may flow through the porous wall 847 from the inner tube flow area 845 into the annular flow area 836 to divide the fluid flow into a third velocity 853 within the inner tube flow area 845 and a fourth velocity 854 within the annular flow area 836. The fourth velocity 854 may be less than the third velocity 853. A portion of the TTM fluid 112 may then flow back into the inner tube flow area 845 from the annular flow area 836 to define a fifth velocity 855 along the inner tube flow area 845 which may be about equal to the second velocity 852. The TTM fluid 112 may then proceed along the nozzle 820 to define a sixth velocity 856 exiting the filter 800. In some embodiments, the first velocity 851 and the sixth velocity 856 may be about equal.

The filter 800 may be configured to remove harmful bacteria and viruses from the TTM fluid 112 using sedimentation principles. In use, the filter 800 may be oriented horizontally so that the direction of fluid flow through the filter 800 is perpendicular to a gravitational force 865. In some instances, bacteria, viruses, and other particles within the TTM fluid 112 may have a greater density than the TTM fluid 112 and as such may be urged by the gravitational force 865 (i.e., sink) in a direction perpendicular to the fluid flow direction. In some instances, particles within the inner tube flow area 845 may sink toward and through the porous wall 847 into the annular flow area 836. Particles within the annular flow area 836 may then sink toward an inside surface 831 of the body 830 and become trapped adjacent the inside surface 831. The geometry of the filter 800 may be configured to allow 0.2-micron bacteria/virus particles to fall out of the flow of TTM fluid 112 and become trapped along the inside surface 831.

In some embodiments, the filter 800 may be configured so that flow of TTM fluid 112 from the inner tube flow area 845 into the annual flow area 836 my drag particles through the porous wall 847. In some embodiments, the inlet flow area 811, the inner tube flow area 845, and the annual flow area 836 may be sized so that the third velocity 853 is less than about 50 percent, 25 percent, or 10 percent of the first velocity 851 or less. In some embodiments, the body 830 and the inner tube 840 may be configured so that the fourth velocity 854 is less than the third velocity 853. In some embodiments, the fourth velocity 854 may less than about 50 percent, 25 percent, or 10 percent of the third velocity 853 or less.

In some embodiments, the filter 800 may be configured so that the flow within the inner tube flow area 845 is laminar flow, i.e., so that the velocity of the fluid flow adjacent to or in close proximity to an inside surface 841 of the porous wall 847 is less than the velocity at a location spaced away from the inside surface 841. In such an embodiment, the particles may more readily sink toward and through the porous wall 847.

In some embodiments, the filter 800 may be configured so that the fluid flow within the annual flow area 836 is laminar flow, i.e., so that the velocity of the fluid flow adjacent to or in close proximity to inside surface 831 of the body 830 is less than the velocity at a location spaced away from the inside surface 831. In such an embodiment, the particles may more readily sink toward and be trapped along the inside surface 831.

The filter 800 may comprise three components including the inner tube 840 an inner body shell 838, and an outer body shell 839. Each of the three components may be formed via the plastic injection molding process. Assembly of the filter 800 may include capturing the inner tube 840 within the inner body shell 838 and the outer body shell 839 and sliding the inner body shell 838 into the outer body shell 839 wherein the fit between the inner body shell 838 and the outer body shell 839 is an interference fit.

In some embodiments, the filter 800 may be disposed within the pad 120. FIG. 8C shows a detail cross-sectional view of the pad 120 including the filter 800 disposed within the fluid containing layer 420. The filter 800 is coupled in line with an internal flow path 860 within the fluid containing layer 420 so that TTM fluid 12 circulating within the pad 120 passes through the filter 800. The filter 800 may be sized so that the inlet flow area 811 and the outlet flow area 821 are similar to a cross-sectional flow area of the internal flow path 860 within the fluid containing layer 420.

In some embodiments, a thickness of the fluid containing layer 420 may increase adjacent the filter 800 to accommodate a body diameter 864 of the filter 800. To further accommodate the body diameter 864, the insulation layer 410 and/or the thermal conduction layer 430 may comprise internal depressions 862, 863, respectively.

In some embodiments, one or more filters 800 may be disposed in line with the flow of TTM fluid 112 at other locations of the TTM system 100. In some embodiments, one or more filters 800 may be disposed within the TTM module 110. In some embodiments, one or more filters 800 may be disposed in line with the FDL 130. In some embodiments, the filter 800 may be disposed in line with a fluid conduit of the pad external to the fluid containing layer 420 such as a conduit extending between the pad connector 652 and the pad 120.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

1. A targeted temperature management (TTM) system, comprising:
a TTM module configured to provide a TTM fluid;
a thermal pad configured to receive the TTM fluid from the TTM module to facilitate thermal energy transfer between the TTM fluid and a patient;
a multi-conduit fluid delivery line extending between the TTM module and the thermal pad, the fluid delivery line configured to provide TTM fluid flow from the TTM module to the thermal pad; and
a connection system configured to provide a connectivity indication to a user, the connection system comprising:
a first connector attached to the fluid delivery line,
a corresponding second connector attached to the thermal pad, and
a connectivity sensor configured to determine if the first connector is connected to the second connector.

2. The system of claim 1, wherein the connectivity indication is a confirming indication that the first connector is completely connected to the second connector.

3. The system of claim 1, wherein the connectivity indication comprises an illuminating indicium.

4. The system of claim 1, wherein the connectivity system comprises one or more indicia indicating proper orientation of the first connector with respect to the second connector.

5. The system of claim 1, wherein the connection system further comprises a connection controller coupled to the connectivity sensor, the connection controller including controller logic.

6. The system of claim 5, wherein the first connector comprises the connection controller.

7. The system of claim 5, wherein the connection system comprises a display coupled to the connection controller, and wherein the controller logic is configured to render the connectivity indication on the display.

8. The system of claim 7, wherein the connection controller and the display are disposed within a controller housing.

9. The system of claim 5, wherein the connection system further comprises a connector lock configured to selectively allow and prevent separation of the first connector from the second connector.

10. The system of claim 9, wherein the connection system further comprises an actuator coupled to the connection controller and the connector lock, and wherein the controller logic is configured to selectively 1) activate the connector lock preventing separation of the first connector from the second connector, and 2) deactivate the connector lock allowing separation of the first connector from the second connector.

11. The system of claim 7, wherein:
the connection system further comprises a flow sensor configured to measure a TTM fluid flow rate;
the flow sensor is coupled to the connection controller; and
the controller logic is configured to render a TTM fluid flow rate indication on the display.

12. The system of claim 11, wherein the controller logic is configured to activate and/or deactivate the lock in accordance with a signal from the flow sensor.

13. The system of claim 7, wherein:
the connection system further comprises a temperature sensor configured to measure a TTM fluid temperature;
the temperature sensor is coupled to the connection controller; and
the controller logic is configured render a TTM fluid temperature indication on the display.

14. The system of claim 5, wherein:
the connection controller is coupled to the TTM module;
the controller logic provides the connectivity indication to the TTM module;
the TTM module comprises a TTM module console including TTM Module logic; and
wherein TTM module logic is configured to selectively allow and prevent TTM fluid flow to the pad in accordance with the connectivity indication.

15. The system of claim 14, wherein the controller logic is configured to provide one or more signals to the TTM module in accordance with signals received from the connection system, temperature sensor, and/or the flow sensor.

16. The system of claim 14, controller logic is configured to render a visualization on the display indicating that separation of the first connector from the second connector is allowed in accordance with a signal received from the TTM module controller.

17. The system of claim 14, wherein the controller logic is configured to activate and/or deactivate the lock in accordance with a signal from the TTM module.

18. The system of claim 1, wherein the fluid delivery line comprises a first conduit and a second conduit, and wherein the first conduit is disposed within second conduit along a length of the fluid delivery line.

19. The system of claim 18, wherein, in use, a direction fluid flow through the first conduit is from the TTM module toward the thermal pad.

20. The system of claim 18, wherein the first conduit is concentrically positioned within the second conduit.

21. The system of claim 20, wherein, the first conduit is attached to the second conduit along the length of the fluid delivery line.

22. The system of claim 1, further comprising a filter disposed in line with a TTM fluid flow path.

23. The system of claim 22, wherein the filter comprises a porous wall disposed parallel to a flow direction of TTM fluid along the TTM fluid flow path.

24. The system of claim 22, wherein the filter is attached to the thermal pad.

25. The system of claim 22, wherein the filter is disposed within a fluid containing layer of the thermal pad.

26. A method of using a targeted temperature management (TTM) system, comprising:
providing a TTM system comprising:
a TTM module configured to provide a TTM fluid;
a thermal pad configured to receive the TTM fluid from the TTM module to facilitate thermal energy transfer between the TTM fluid and a patient;
a multi-conduit fluid delivery line (FDL) extending between the TTM module and the thermal pad, the FDL configured to provide TTM fluid flow from the TTM module to the thermal pad; and
a connection system comprising:
an FDL connector attached to the FLD; and
a pad connector attached to the thermal pad,
wherein the FDL connector is configured to connect with the pad connector to establish fluid communication between the FDL and the thermal pad; and
a connectivity system configured to provide a visual indication to the user when the FDL connector is connected to the pad connector;
connecting the FDL connector with the pad connector;
observing the visual indication that the FDL connector is connected to the pad connector; and
delivering TTM fluid from the TTM module to the thermal pad.

27. The method of claim 26, wherein the connection system further comprises a latching system configured to inhibit separation of the FDL connector from the pad connector, the method further comprising latching the FDL connector to the pad connector.

28. The method of claim 26, wherein the connection system further comprises a locking system configured to prevent separation of the FDL connector from the pad connector by a user, the method further comprising locking the FDL connector to the pad connector.

29. The method of claim 26, wherein the connection system further comprises a connection controller, and wherein observing the visual indication comprises observing the visual indication on a display of the controller.

30. The method of claim 29, wherein the connection controller is coupled to the TTM module, and wherein observing the visual indication comprises observing the visual indication on a display of the TTM module.

31. The method of claim 29, wherein the locking system comprises a locking actuator coupled to the connection controller, and wherein locking the FDL connector to the pad connector comprises activating of the locking actuator by the connection controller.

32. The method of claim 31, wherein the connection controller is coupled to the TTM module, and wherein locking the FDL connector to the pad connector comprises activating the locking actuator in response to signal from the TTM module.

33. The method of claim 32, wherein the connection controller is coupled to the TTM module, and wherein delivering TTM fluid is performed after activating the locking actuator.

34. A medical pad for facilitating thermal energy transfer between a targeted temperature management (TTM) fluid and a patient, the pad comprising:
a fluid containing layer; and
a fluid connector in fluid communication with the fluid containing layer, the fluid connector configured to couple with a corresponding fluid line connector attached to a TTM fluid delivery line,
wherein:
the fluid connector comprises a pad component of a connectivity system,
the pad component is configured to interact with a corresponding fluid line component of the connectivity system when the fluid connector is coupled with the corresponding fluid line connector, and
the connectivity system is configured to provide an indication to a user that the fluid connector is coupled with the corresponding fluid line connector.
